(19) United States
(12) Patent Application Publication
(10) Pub. No.: US 2023/0302411 A1
(43) Pub. Date: Sep. 28, 2023

(54) ASYMMETRIC HYDROPHOBIC POLYOLEFIN HOLLOW FIBER MEMBRANE, PREPARING METHOD, AND USE OF THE SAME

(71) Applicant: HANGZHOU COBETTER TECHNOLOGY CO., LTD., Hangzhou, Zhejia (CN)

(72) Inventors: Jiandong JIA, Hangzhou (CN); Mengze CHEN, Hangzhou (CN); Jiadai YU, Hangzhou (CN)

(73) Assignee: HANGZHOU COBETTER TECHNOLOGY CO., LTD., Hangzhou, Zhejia (CN)

(21) Appl. No.: 18/021,074

(22) PCT Filed: Aug. 13, 2021
(86) PCT No.: PCT/CN2021/112498

§ 371 Date: Feb. 13, 2023

(30) Foreign Application Priority Data
Aug. 17, 2020 (CN) 202010827754.X

(51) Int. Cl. B01D 69/08; B01D 69/02; B01D 71/26; B01D 53/22; A61M 1/16; B01D 67/00

(52) U.S. Cl. CPC B01D 69/082; B01D 69/02; B01D 71/26; B01D 53/228; A61M 1/16; B01D 67/0011; B01D 67/0083; B01D 2325/022; B01D 2325/38; A61M 2202/0241; B01D 2325/02833; B01D 2325/02834; B01D 2325/24; B01D 2325/04; B01D 2325/20; B01D 2323/082; B01D 2323/22; B01D 2323/18; B01D 2323/12; B01D 2323/081; B01D 2323/14

(57) ABSTRACT

An asymmetric hydrophobic polyolefin hollow fiber membrane includes a support layer and a separation layer, the separation layer including an outer surface, the outer surface including a quantity of first pores with a certain pore size; presence of the first pores facilitates an anesthetic gas such as sevoflurane and remifentanil to permeate through the hollow fiber membrane into the human blood, allowing for the patient to maintain sedated throughout a surgical process; meanwhile, the first pores facilitate reduction of dosage of the anesthetic in the surgery, thereby reducing surgical costs and avoid overdosage of the anesthetic causing secondary impairment to the patient; in addition, the hollow fiber membrane offers a long plasma permeation duration, a high tensile strength and a high elongation at break to satisfy application needs, particularly suitable for human blood oxygenation including anesthetic gas and the gas-liquid separation areas.

### FIELD

Embodiments of the disclosure relate to membrane materials, and more particularly relate to an asymmetric hydrophobic polyolefin hollow fiber membrane, a preparing method, and use of the same.

### BACKGROUND

Various applications in chemical, biochemical or medical fields require separating a gaseous component from a liquid or adding a gaseous component to a liquid. For such gas exchange processes, membranes are increasingly used. A separation membrane is disposed between a liquid and a fluid which adsorbs or releases a gaseous component, serving to separate the gaseous component from the liquid, or add the gaseous component to the liquid. The fluid here may refer to a gas or may refer to a liquid comprising or adsorbing a to-be-exchanged gaseous component. Such membranes may provide an exchange surface for gas exchange and if necessary, may prevent direct contact between the liquid and the fluid.

An important application of a membrane-based gas exchange process in the medical field is an oxygenator, which is also referred to as an artificial lung. The oxygenator is applied, for example, in an open-heart surgery for blood oxygenation and/or for removing carbon dioxide from the blood. The oxygenator generally uses a bundled hollow fiber membrane, wherein the venous blood flows through the outer space surrounding the hollow fiber membrane, and the lumen of the hollow fiber membrane is charged with air, oxygenized air, or even pure oxygen. This hollow fiber membrane allows oxygen to access the blood while removing carbon dioxide from the blood into the gas in the lumen.

Presently, the hollow fiber membranes used in oxygenators are mostly asymmetric membranes comprising a separation layer and a support layer, wherein the support layer has a relatively high porosity to ensure that the oxygen and carbon dioxide can permeate through the membrane more freely, i.e., providing a relatively high permeation rate for both oxygen and carbon dioxide; while the separation layer is a dense layer, i.e., without pores at the outer surface of the separation layer, which ensures that the hollow fiber membrane has a relatively long plasma permeation duration and a relatively long service life (at least over 48 hours), eliminating a need of changing the hollow fiber membrane in the middle of a surgery, thereby avoiding the impact on surgery success due to changing the hollow fiber membrane.

In various medical surgeries, patients are required to fall asleep for relaxation purposes; therefore, it is desirable to add an anesthetic (i.e., an anesthetic gas) into the air breathed so as to sedate the patients. However, since the separation layer of a conventional hollow fiber membrane is a dense layer without pores at the outer surface, the anesthetic gas can hardly permeate through the hollow fiber membrane into the human blood. To ensure a successful surgery, the patient is needed to receive an excessive dosage of anesthetic before the surgery so as to maintain asleep throughout the surgical process. However, since such anesthetics are generally very expensive, application of an excessive dosage of an anesthetic will significantly increase surgery costs, aggravating the patient's economic burden; in addition, the excessive dosage of anesthetic is prone to cause secondary impairments to the patient's physical and psychological health. Therefore, it is needed to develop a hollow fiber membrane which allows permeation of an anesthetic gas and has a long plasma permeation duration and a strong mechanical property.

### SUMMARY

To overcome the above and other drawbacks in the prior art, the present disclosure provides an asymmetric hydrophobic polyolefin hollow fiber membrane, wherein an outer surface of a separation layer of the hollow fiber membrane has a quantity of first pores with a certain pore size, allowing for an anesthetic gas to permeate through the hollow fiber membrane into a patient's blood at a predetermined permeation rate such that the patient maintains asleep throughout a surgical process; meanwhile the hollow fiber membrane has a long plasma permeation duration and a strong mechanical property.

According to some embodiments of the present disclosure, there is provided an asymmetric hydrophobic polyolefin hollow fiber membrane, comprising a support layer and a separation layer, the support layer comprising an inner surface facing a lumen of the hollow fiber membrane, the separation layer comprising an outer surface, the outer surface being located at the side of the separation layer opposite the support layer, wherein: the outer surface comprises a plurality of first pores, the first pores having a pore size of 10 nm to 300 nm in a first direction of the outer surface and a pore size of 10 nm to 300 nm in a second direction of the outer surface; wherein the first direction of the outer surface is parallel to an axial direction of the hollow fiber membrane, and the second direction of the outer surface is parallel to a radial direction of the hollow fiber membrane; the first pores at the outer surface have a pore density of 4 to 45 pores/1 μm²; the outer surface of the hollow fiber membrane has a surface energy of 10 mN/m to 45 mN/m under 20° C.; the hollow fiber membrane has a tensile strength of at least 100CN and an elongation at break of at least 150%.

The hollow fiber membrane of the present disclosure is made of a polyolefin substance, which only comprises carbon and hydrogen, without other elements. The hollow fiber membrane is an asymmetric membrane, comprising a support layer and a separation layer, wherein the support layer has an open micro-porous structure; the support layer does not have large pores, and the pores at the support layer are uniform and substantially isotropous. The support layer and the separating layer are made of the same material; the two layers are bonded into an integral structure but have different membrane structure properties such as thickness. The separation layer comprises an outer surface which is located at the side of the separation layer opposite the support layer. Different from the outer surface of a conventional hollow fiber membrane (where no pores are provided at the outer surface), the outer surface of the hollow fiber membrane according to the present disclosure comprises a quantity of first pores with a certain pore size. If the pore size of the first pores is too small, the anesthetic gas cannot permeate through the hollow fiber membrane into a patient's blood such that the patient cannot be surely sedated throughout the surgical process. If the pore size of the first pores is too large, the plasma permeation duration of the hollow fiber membrane would be significantly decreased, while a too short service life cannot satisfy surgical needs. If the number of the first pores at the outer surface is too small, the permeation rate of the anesthetic gas would be very slow; if the number of the first pores is too large, the surgery will consume much anesthetic gas, which increases surgical costs and also significantly reduces the plasma permeation duration of the hollow fiber membrane. Therefore, to ensure that the hollow fiber membrane has a relatively long plasma permeation duration and the anesthetic gas can permeate through the hollow fiber membrane into the patient's blood at an appropriate permeation rate, an appropriate quantity of first pores with an appropriate pore size need to be provided at the outer surface. In the present disclosure, the first pores have a pore size of 10 nm to 300 nm in a first direction of the outer surface and a pore size of 10 nm to 300 nm in a second direction of the outer surface; wherein the first direction of the outer surface is parallel to the axial direction (the lengthwise direction of the hollow fiber membrane) of the hollow fiber membrane, and the second direction of the outer surface is parallel to the radial direction of the hollow fiber membrane. The pore size of the first pores in the first direction may be identical to the pore size in the second direction or may not be identical to the pore size in the second direction, i.e., the first pores may have an oval or round shape. The first pores at the outer surface have a pore density of 4-45 pores/1 μm². Since a certain quantity of first pores with a certain pore size are provided at the outer surface of the hollow fiber membrane in the present disclosure, an anesthetic gas such as sevoflurane, xenon, remifentanil or propofol may permeate through the hollow fiber membrane into the patient's blood at an appropriate permeation rate, such that the patient maintains asleep throughout the surgical process, which ensures smooth proceeding of the surgery; since the patient maintains asleep without being administered with excessive dosage of anesthetic gas before the surgery, the dosage of the anesthetic gas applied during the surgery would be significantly reduced, which not only reduces the economic cost and mitigates the patient's economic burden on one hand, but also avoids secondary impairment to the patient's physical and psychological health due to application of excessive dosage of anesthetic gas. However, the certain quantity of first pores with a certain pore size at the outer surface would have an impact on the plasma permeation duration of the hollow fiber membrane; however, the inventors of the disclosure surprisingly find that presence of the quantity of pores with a certain pore size at the outer surface has less impact on the plasma permeation duration of the hollow fiber membrane, and the hollow fiber membrane still has a relatively long plasma permeation time and a relative long service life to satisfy surgical needs.

To measure the pore size and pore density of the first pores at the outer surface of the hollow fiber membrane, a SEM (Scan Electron Microscopy) is used to characterize the morphology of the membrane structure, and then the morphological characteristics of the hollow fiber membrane are measured using computer software (e.g., Matlab, NIS-Elements, etc.) or manually, followed by corresponding computation. During the membrane preparing process, various features such as pore size distribution are substantially uniform and consistent in the direction perpendicular to the membrane thickness direction (in the disclosure, since the membrane has a hollow fiber membrane morphology, the direction is perpendicular to the radius direction); therefore, the pore size in a partial region on a corresponding plane may be used to reflect the pore size on the overall plane. In actual measurement, the outer surface of the membrane may be first characterized using the SEM to obtain a corresponding SEM image; since the pores at the outer surface of the membrane are substantively uniform, a certain area, e.g., 1 μm² (1 μm*1 μm), may be selected (the specific area size is selected dependent on actual conditions), and then the pore size of all pores in the area may be measured using computer software or manually, followed by computation to obtain the pore density of the surface. It is noted that the measurement methods described herein are only for reference, and those skilled in the art may obtain the parameters via other measurement methods.

The surface energy of water is 72.8 mN/m at 20° C. If the surface energy of the membrane is lower than 72.8 mN/m, it indicates that the outer surface of the membrane has a certain hydrophobic property; the greater the hydrophobic property of the membrane is, the longer plasma permeation duration the membrane has. The outer surface of the hollow fiber membrane according to the present disclosure has a surface energy of 10 to 45 mN/m at 20° C., which indicates that the outer surface of the membrane has a relatively strong hydrophobic property, further indicating that the hollow fiber membrane according to the present disclosure has a relatively long plasma permeation duration, thereby satisfying surgical needs. To measure the surface energy of the outer surface of the hollow fiber membrane, a Dyne pen is utilized, wherein the Dyne pen is drawn over the outer surface of the hollow fiber membrane in a 10 cm-long ink pass and then it is observed whether over 90% of the ink pass has been drawn back into droplets in less than 2 s till the ink pass stops drawback and droplets emerge; the measured surface energy of the ink is the surface energy of the outer surface of the membrane. In addition, a certain quantity of first pores with a certain pore size at the outer surface likely have an impact on the mechanical strength of the hollow fiber membrane; however, the inventors of the disclosure surprisingly find that presence of the certain quantity of first pores with a certain pore size in the disclosure has less impact on the mechanical strength of the hollow fiber membrane. The hollow fiber membrane according to the present disclosure has a tensile strength of at least 100CN and an elongation at break of at least 150%, which indicates that the hollow fiber membrane according to the present disclosure still has a relatively high mechanical strength, further indicating that it has a large industrial value and can satisfy manufacturing requirements. To measure the tensile strength and the elongation at break, the hollow fiber membrane according to the present disclosure is stretched by a stretcher at a uniform speed under the room temperature (wherein the stretching speed is 50 mm/min and the distance between the upper and lower fixtures is 30 mm) till being broken, whereby the tensile strength and the elongation at break are measured. The testing is repeated three times, with their mean values being computed, wherein the mean tensile strength and the mean elongation at break are the final tensile strength and the final elongate at break of the hollow fiber membrane.

As a further improvement provided by the present disclosure, the outer surface comprises a plurality of first pores, wherein the first pores have a pore size of 150 nm to 300 nm in the first direction of the outer surface, the first pores have a pore size of 10 nm to 90 nm in the second direction of the outer surface; the first direction is parallel to the axial direction of the hollow fiber membrane, and the second direction is parallel to the radial direction of the hollow fiber membrane; and the first pores have a pore density of 4 to 35 pores/1 μm².

In the case that the pore size of the first pores in the first direction of the outer surface (i.e., the axial direction of the hollow fiber membrane) is 150 nm-300 nm and the pore size in the second direction of the outer surface (i.e., the radial direction of the hollow fiber membrane) is 10 nm-90 nm, the first pores have an elongated oval shape; and the pore density of the first pores is 4-35 pores/1 μm². With this shape, the quantity of first pores may facilitate the anesthetic gas to permeate through the hollow fiber membrane into a patient's blood at a certain permeation rate, substantially not affecting the plasma permeation duration of the hollow fiber membrane, such that the hollow fiber membrane still has a relatively long plasma permeation duration.

As a further improvement provided by the present disclosure, the separation layer has a thickness of 0.1 μm to 2 μm, accounting for 0.5 to 5% of total thickness of the hollow fiber membrane. An overly large separation layer thickness would significantly increase the time for the oxygen and the carbon oxide to permeate through the hollow fiber membrane such that the carbon dioxide cannot be discharged out from the blood in time and the oxygen cannot access the blood in time, which would affect smooth proceeding of the surgery; while an overly small separation layer thickness would significantly reduce the plasma permeation duration such that the service life of the hollow fiber membrane is significantly shortened. In the present disclosure, the separation layer has a thickness of 0.1 μm to 2 μm, accounting for 0.5 to 5% of total thickness of the hollow fiber membrane. The appropriate thickness of the separation layer ensures a relatively short time for the oxygen and carbon dioxide to permeate through the hollow fiber membrane, not affecting normal proceeding of the surgery, whereby life and health of the patient are ensured; meanwhile, the appropriate thickness of the separation layer enables the hollow fiber membrane to have a relatively long plasma permeation duration, such that the hollow fiber membrane has a relatively long service life. The thickness of the separation layer and the total thickness of the hollow fiber membrane may be measured by using a SEM to characterize the morphology of the hollow fiber membrane structure and then computing manually or with computer software (e.g., Matlab, NIS-Elements, etc.). It is noted that the measurement means described above are only for reference, and those skilled in the art may also obtain the parameters via other measurement methods.

As a further improvement provided by the present disclosure, the separation layer is porous, and a mean pore size of the separation layer is 10 nm to 60 nm. Conventional hollow fiber membranes have a dense layer without pores in the inside. However, the hollow fiber membrane according to the present disclosure is porous, i.e., pores are present in the inside of the separation layer, such that there is a certain porosity. Presence of pores inside the separation layer facilitates an anesthetic gas to permeate through the hollow fiber membrane into a patient's blood, such that the patient would maintain asleep throughout the surgical process. However, if the pore size of the pores inside the separation layer is overly large, the plasma permeation duration of the hollow fiber membrane would be reduced such that the hollow fiber membrane cannot satisfy surgical needs. In the present disclosure, the mean pore size of the separation layer of the hollow fiber membrane is 10 nm-60 nm, preferably 20-50 nm, which not only ensures permeation of the anesthetic gas through the hollow fiber membrane at a certain permeation rate, but also enables the hollow fiber membrane to have a relatively long plasma permeation duration, i.e., a relatively long service life. The mean pore size of the separation layer may be measured using bubble point method, mercury porosimetry or other measurement methods.

As a further improvement provided by the present disclosure, the hollow fiber membrane has an O₂ permeation rate of 1 to 50 L/min·bar·m²; the hollow fiber membrane has a gas separation factor α of 1 to 4 between CO₂ and O₂ and a gas separation factor α of at least 150 between O₂ and an anesthetic gas.

In the present disclosure, the hollow fiber membrane has an O₂ permeation rate of 1 to 50 L/min·bar·m², which shows that the hollow fiber membrane according to the present disclosure has a relatively large oxygen permeation rate, such that the oxygen in the lumen may access the patient's blood through the hollow fiber membrane in a short time, thereby ensuring smooth breathing of the patient. The separation factor refers to a ratio between permeation rates of two gases; in the present disclosure, the hollow fiber membrane has a gas separation factor α of 1 to 4 between CO₂ and O₂, indicating that compared with the oxygen permeation rate, the hollow fiber membrane according to the present disclosure has a higher carbon dioxide permeation rate, which facilitates quick discharge of the carbon dioxide from the blood and does not cause secondary impairment to the patient's physical and psychological health; this not only ensures smooth proceeding of the surgery but also ensures physical and psychological health of the patient. Before the surgery, a medical staff would inject a certain amount of anesthetic into the patient's body to make the patient asleep; during the surgical process, to maintain the patient asleep, a certain amount of anesthetic gas is needed to permeate through the hollow fiber membrane into the patient's blood; however, the amount of the anesthetic gas needed during the surgical process is very small. In the present disclosure, the hollow fiber membrane has a separation factor of over 150 between the oxygen and the anesthetic gas, which, on one hand, indicates that the anesthetic gas may permeate through the hollow fiber membrane into the patient's blood, and on the other hand, indicates that hollow fiber membrane has a very low anesthetic gas permeation rate. During the surgical process, only a very small amount of anesthetic gas may penetrate through the hollow fiber membrane into the patient's blood, which may not only ensure that the patient maintains asleep throughout the surgical process, but also ensures that only a small amount of anesthetic gas is dosed during the surgical process such that the surgical cost is low and no secondary impairment to the patient's health is caused.

The gas permeation rate of the hollow fiber membrane (for oxygen, carbon dioxide, or other gas) is tested using the following method: subjecting one side of a membrane sample of 0.1 m² to a to-be-tested gas (oxygen, carbon dioxide or other gas) at 25° C. under 1bar; feeding the to-be-tested gas into the lumen of the hollow fiber membrane; measuring the volumetric flow rate of the to-be-tested gas permeated through the sample membrane wall using a flowmeter; repeating the measurement three times from the inside of the membrane to the outside of the membrane and three times from the outside of the membrane to the inside of the membrane, and averaging the measurements, wherein the resultant mean value is the permeation rate of the membrane with respect to the to-be-tested gas.

As a further improvement of the present disclosure, the hollow fiber membrane has an O₂ permeation rate of 10 to 40 L/min·bar·m² and a CO₂ permeation rate of 15 to 80 L/min·bar·m².

The CO₂ permeation rate of the hollow fiber membrane in the present disclosure is 15-80 L/min·bar·m², indicating that the hollow fiber membrane according to the present disclosure has a large CO₂ permeation rate such that CO₂ in the blood can be quickly discharged, which causes no impact on the physical and psychological health of the patient and facilitates smooth proceeding of the surgery.

As a further improvement provided by the present disclosure, the hollow fiber membrane has a gas separation factor α of at least 200 between O₂ and the anesthetic gas, wherein the anesthetic gas is selected from the group consisting of at least one of sevoflurane, xenon, remifentanil, and propofol. When the anesthetic gas is at least one of sevoflurane, xenon, remifentanil, and propofol, the separation factor of the hollow fiber membrane between oxygen and the anesthetic gas is over 200, which, on one hand, indicates that the anesthetic gas can permeate through the hollow fiber membrane into the patient's blood, and on the other hand, indicates that the hollow fiber membrane has a very low permeation rate with respect to the anesthetic gas, such that during the surgical process, only a very small amount of anesthetic gas may permeate through the hollow fiber membrane into the patient's blood; this not only ensures that the patient maintains asleep during the surgical process, but also ensures that only a small amount of anesthetic gas is dosed during the surgical process, which leads to a low surgical cost and causes no secondary impairment to the patient's health.

As a further improvement provided by the present disclosure, the hollow fiber membrane has a plasma permeation duration of at least 48 h.

In the present disclosure, the plasma permeation duration of the hollow fiber membrane is at least 48 h, indicating that the hollow fiber membrane has a relatively long service life, eliminating a need of changing the hollow fiber membrane during various surgical operations, which ensures normal proceeding of the surgery and mitigates the impact of external factors on surgery success.

As a further improvement provided by the present disclosure, the hollow fiber membrane further comprises a transition layer disposed between the support layer and the separation layer, the transition layer having a thickness of 10 nm to 50 nm and a mean pore size of 100 nm to 300 nm. In the present disclosure, the hollow fiber membrane according to the present disclosure further comprises a very thin transition layer between the support layer and the separation layer, wherein the thickness of the transition layer is only 10 nm to 50 nm and the mean pore size thereof is 100 nm to 300 nm. The transition layer is a transitional area between the support layer and the separation layer; presence of the transition layer indicates that the transition of the membrane structure from the separation layer to the support layer is not abrupt, but progressive. Presence of the transition layer increases the bonding strength between the support layer and the separation layer such that the bonding becomes tighter, thereby increasing the tensile strength and the elongation at break of the hollow fiber membrane, such that the hollow fiber membrane has a wide range of applications.

As a further improvement provided by the present disclosure, the hollow fiber membrane has a thickness of 30 μm to 50 μm and an inner diameter of 100 μm to 300 μm; and the hollow fiber membrane has a volumetric porosity of 30% to 60%. A too-small membrane thickness would affect the tensile strength of the membrane while a too-large thickness of the membrane would affect the time taken by the gases such as oxygen and carbon dioxide to permeate through the membrane. In the present disclosure, the thickness of the hollow fiber membrane is 30 μm to 50 μm, which not only ensures that the hollow fiber membrane has a relatively high tensile strength but also enables the gases such as oxygen and carbon dioxide to permeate through the membrane in a shorter time, thereby enabling the carbon dioxide in the blood to quickly discharged and enabling the oxygen to access the blood quickly. The inner diameter of the hollow fiber membrane is 100 μm to 300 μm, which ensures enough oxygen to flow into the inner diameter of the membrane and then reaches the human blood, thereby ensuring smooth proceeding of the surgery. An overly high porosity of the membrane would affect the tensile strength of the membrane; while an overly low porosity of the membrane would affect the permeation rate of the oxygen and carbon dioxide. In the present disclosure, the volumetric porosity of the hollow fiber membrane is 30% to 60%, which not only ensures a relatively large tensile strength of the hollow fiber membrane, but also provides a relatively high oxygen permeation rate and a relatively high carbon dioxide permeation rate. In the present disclosure, for the thickness of the inner diameter of the hollow fiber membrane may be obtained by using a SEM to characterize the morphology of the membrane structure and then measure manually or using computer software (e.g., Matlab, NIS-Elements, etc.); the volumetric porosity of the membrane may be obtained by mercury porosimetry using a mercury porosimeter.

The present disclosure further provides a method of preparing an asymmetric hydrophobic polyolefin hollow fiber membrane, comprising steps of:

step 1: heating to plasticize a polyolefin polymer comprising only carbon and hydrogen, followed by dissolving the plasticized polyolefin polymer in a solvent system comprising compound A and compound B, and mixing under an environment higher than a critical delamination temperature, resulting in a homogeneous casting solution, wherein compound A is a solvent for polyolefin polymer, and compound B is a non-solvent for polyolefin polymer and elevates the separation temperature for a phase comprising the polyolefin polymer and the compound A; the solvent system has a range exhibiting a homogeneous solution at an elevated temperature, a critical delamination temperature upon cooling, a miscibility gap in a liquid state of aggregation lower than the critical delamination temperature, and a cold curing temperature;

step 2: extruding the casting solution in a die having a temperature higher than the critical delamination temperature to form a molding having an inner surface and an outer surface;

step 3: placing the molding in an air section for preliminary phase separation;

step 4: cooling the molding with a coolant comprising compound A at a cooling temperature of 5° C. to 60° C. for 20 ms to 75 ms;

step 5: quenching the molding with a quenchant comprising compound A at a quenching temperature of 40° C. to 80° C. for 2 h to 5 h, whereby a nascent membrane is obtained upon end of the quenching;

step 6: removing compound A and compound B from the nascent membrane to obtain a prototype membrane.

As a further improvement provided by the present disclosure, the polyolefin polymer is selected from the group consisting of at least one of polyethylene, polypropylene, and poly(4-methyl-1-pentene); and a concentration of the polyolefin polymer in the casting solution is 30% to 50%.

As a further improvement provided by the present disclosure, the compound A is selected from the group consisting of one or more of dehydrated castor oil fatty acid, methyl-12-hydroxystearate, paraffin oil, dibutyl sebacate, and dibutyl phthalate; the compound B is selected from the group consisting of one or more of dioctyl adipate, castor oil, mineral oil, palm oil, rapeseed oil, olive oil, dimethyl phthalate, dimethyl carbonate, and glyceryl triacetate; and a mass ratio of compound A to compound B is 1-5:1.

As a further improvement provided by the present disclosure, in step 3, the molding stays in the air section for 1.5 ms to 20 ms; the air section has a temperature of 50° C. to 150° C. and a relative humidity of not greater than 50%.

The present disclosure utilizes a thermally induced phase separation method to prepare a hollow fiber membrane. When preparing the hollow fiber membrane, the first step is to plasticize the polyolefin substance. Plasticization refers to a process of heating a polyolefin substance in a solution tank to a flowable state with a good plasticity, and the purpose of plasticizing the polyolefin substance is to homogeneously disperse the polyolefin substance in the solvent system comprising compound A and compound B so as to form a homogeneous solution, facilitating obtaining a hollow fiber membrane with a good integrity. In the present disclosure, the polyolefin substance is selected from the group consisting of one or more of polyethylene, polypropylene, and poly(4-methyl-1-pentene). Such substances are nontoxic and harmless and meanwhile have a good biocompatibility. The resultant hollow fiber membrane has a high gas (oxygen, carbon dioxide) permeation rate and a good mechanical property. The plasticized polyolefin substance is dissolved in a solvent system comprising compound A and compound B and mixed under an environment higher than the critical delamination temperature to obtain a homogeneous casting solution, wherein compound A is a solvent for the polyolefin polymer. The solvent for polymer means when being heated at most to the boiling point of compound A, compound A may dissolve the polyolefin polymer to form a homogeneous solution. In the present disclosure, compound A is selected from the group consisting of one or more of dehydrated castor oil fatty acid, methyl-12-hydroxystearate, paraffin oil, dibutyl sebacate, and dibutyl phthalate; while compound B is a non-solvent for polyolefin polymers. A non-solvent for polymer means when being heated at most to the boiling point of the compound, the compound does not dissolve the at least one of the polymers to form a homogeneous solution. In the present disclosure, compound B is selected from the group consisting of one or more of dioctyl adipate, castor oil, mineral oil, palm oil, rapeseed oil, olive oil, dimethyl phthalate, dimethyl carbonate, and glyceryl triacetate; and a mass ratio of compound A to compound B is 1-5:1. Compound B elevates the separation temperature of the phase comprising the polyolefin polymer and compound A, while addition of compound B facilitates controlling the properties such as pore size of the resultant hollow fiber membrane. In the resultant mixture of casting solution, the mass ratio of the polyolefin polymer is 30% to 50%, and the mass ratio of the solvent system comprising compound A and compound B is 70% to 50%; particularly preferably, the mass ratio of the polymer is 35% to 45%, and the mass ratio of the solvent system is 65% to 55%. The membrane prepared by the solvent system exhibits the desired characteristics with respect to gas permeation rate and selectivity and also exhibits a good mechanical property. Of course, if necessary, an alternative substance such as an antioxidant, a nucleating agent, a filler, or a similar substance may be used as an additive to the polyolefin polymer, compound A, compound B, or the polymer solution.

The second step is to extrude the casting solution in a die with a temperature higher than the critical delamination temperature to form a molding having an inner surface and an outer surface. The molding is a hollow fiber membrane. The casting solution is extruded through a middle porous cavity of the hollow fiber die, wherein the middle porous cavity serves as an inner core to form and stabilize the lumen of the hollow fiber membrane. During the extrusion process, the inner core is heated to a temperature substantially identical to the polymer solution. The extruded hollow fiber membrane has a surface facing the lumen, i.e., inner surface, and a surface opposite the lumen, i.e., outer surface, the outer surface being spaced from the inner surface by a hollow fiber membrane wall. In the present disclosure, the inner core adopted in extruding the hollow fiber membrane is of a gas form, selected from the group consisting of nitrogen, agon, or another inert gas, so as to ensure that the intensity of pressure inside the lumen of the hollow fiber membrane maintains equilibrium with the external intensity of pressure, thereby stabilizing the lumen of the hollow fiber membrane.

The third step is to subject the molding to pass through an air section for preliminary phase separation; the molding stays in the air section for 1.5 ms to 20 ms, wherein the air section has a temperature of 50° C. to 150° C. and a relative humidity of not greater than 50%; preferably, the molding stays in the air section for Sins to 15 ms, wherein the air section has a temperature of 75° C. to 125° C. and a relative humidity of 15% to 45%;

When the system temperature is greater than or equal to the critical delamination temperature, the polyolefin polymer, compound A, and compound B may form a single-phase, homogeneous solution; as the system temperature drops, liquid-liquid delamination occurs to the homogeneous solution, wherein the two liquids are coexistent, i.e., one phase with a high polymer content and one phase with a low polymer content appear, such that when the temperature further drops, a cold curing phenomenon occurs. The air section in the present disclosure refers to a gas atmosphere comprising air or nitrogen or argon or another inert gas. In the air section, initialization of liquid-liquid phase separation occurs to the homogeneous solution, which facilitates evaporation of compound B in the phase with a low polymer content (i.e., separation layer), resulting in initialization of preliminary phase separation of the molding. By adjusting the temperature of the air section and the stay time of the molding in the air section, a hollow fiber membrane with a desired separation layer (having pores with a certain pore size) is obtained, and meanwhile the separation still allows for sufficient permeability of carbon dioxide and oxygen and a relatively long plasma permeation duration.

The fourth step is to cool the phase pre-separated molding using a coolant, wherein the cooling temperature is 5° C. to 60° C. and the stay time for cooling is 20-75 ms; preferably, the cooling temperature is 20° C. to 50° C. and the stay time for cooling is 35 ms to 65 ms. The coolant may only comprise compound A or a mixture of compound A and compound B; when subjecting the molding to phase separation and curing, selection of coolant type, appropriate cooling temperature, and appropriate length of stay time is crucial, which dictates whether a hollow fiber membrane with an ideal membrane structure may be finally obtained. In the present disclosure, in order to obtain a final hollow fiber membrane having a quantity of first pores with a certain pore size at the outer surface, it is needed to adjust the phase-separation and curing speed (cooling speed), because a too fast phase-separation and curing speed would leave no pores at the outer surface, resulting in a dense separation layer, which is adverse to permeation of anesthetic gas; while a too slow phase-separation and curing speed (cooling speed) would leave pores with a larger pore size at the outer surface, which significantly reduces the plasma permeation duration of the hollow fiber membrane, not satisfying the surgical needs. However, the inventors find in their researches that if the coolant comprises only non-solvent compound B, the phase-separation and curing speed (cooling speed) of the molding would be too fast, resulting a dense separation layer for the final hollow fiber membrane; without pores at the outer surface, the anesthetic gas cannot permeate through the hollow fiber membrane to access the patient's blood, and such a membrane structure is not desired. Therefore, the coolant used in the cooling procedure shall select compound A or a mixture comprising compound A and compound B so as to obtain a hollow fiber membrane with a desired membrane structure, which is totally different from conventional hollow fiber membrane preparing methods that mostly use non-solvent compound B as the coolant.

The fifth step is to subject the molding to quenching treatment using a quenchant, wherein the quenching temperature is 40° C. to 80° C. and the quenching duration is 2 h to 5 h; preferably, the quenching temperature is 50° C. to 70° C. and the quenching duration is 2.5 h to 4.5 h. The quenchant may only comprise compound A or may comprise a mixture of compound A and compound B; the quenching may eliminate membrane stress on one hand and allows for the quenched nascent membrane to have a certain strength; without the quenching procedure, when the membrane is wound, it is likely stretched or even broken, affecting the quality of the final membrane material.

Step 6 is to remove compound A and compound B from the nascent membrane to obtain a prototype membrane. The removal may be implemented by for example extracting. The extraction liquid may be selected from the group consisting of acetone, methanol, and ethanol, preferably isopropanol. The resultant hollow fiber membrane has a porous separation layer, the outer surface of the separation layer having a quantity of pores with a certain pore size, which facilitates the anesthetic gas to permeate through the hollow fiber membrane into the patient's blood at a certain permeation rate, thereby sedating the patient throughout the surgical process.

As a further improvement provided by the present disclosure, before the molding is subjected to cooling treatment in step 4, the molding resulting from preliminary phase separation in step 3 is pre-cooled with a treating solution comprising compound A at a precooling temperature ranging from 120° C. to 160° C. for a precooling duration of 2 ms to 10 ms.

Preferably, the precooling temperature is 130° C. to 150° C. and the precooling duration is 4 ms to 8 ms; the treatment solution may only comprise compound A or may comprise a solvent system comprising compound A and compound B;

In order for the transition of the membrane structure from the support layer to the separation layer not to be abrupt, but progressive, the present disclosure may configure a precooling treatment step after the molding passes through the air section but before the cold curing procedure. The precooling temperature is higher than the cooling temperature, but the precooling duration is shorter than the cooling treatment duration, which facilitates generation of a thin transition layer between the support layer and the separation layer after the precooling treatment. Presence of the transition layer increases the bonding strength between the separation layer and the support layer and increases the mechanical strength of the hollow fiber membrane; moreover, the thin transition layer will not affect the overall structure of the membrane, such that the hollow fiber membrane still has a high gas (oxygen, carbon dioxide) permeation rate.

As a further improvement provided by the present disclosure, the prototype membrane resulting from step 5 is placed under an environment of 120° C. to 180° C. for high-temperature setting and stretched by 0.5% to 10% to relieve stress, whereby a finished membrane is obtained.

Preferably, the prototype membrane is placed under an environment of 135° C. to 165° C. for high-temperature setting and stretched by 2% to 8%;

After high-temperature setting and stretching treatment, the resultant finished membrane has a high tensile strength and a high elongation at break so as to be able to satisfy practical needs of industrial manufacturing.

As a further improvement provided by the present disclosure, a use of an asymmetric hydrophobic polyolefin hollow fiber membrane is provided, wherein the hollow fiber membrane is used for human blood oxygenation comprising an anesthetic gas. In the present disclosure, the hollow fiber membrane has a quantity of first pores with a certain pore size at the outer surface of the separation layer, such that the anesthetic gas can permeate into the patient's blood at a certain permeation rate, allowing for the patient to maintain asleep throughout the whole surgical process, eliminating a need to apply an excessive dosage of anesthetic to the patient before surgery, which not only ensures smooth proceeding of the surgery, but also reduces dosage of the anesthetic gas during the surgery, such that the surgical cost is reduced and secondary impairments to the patient's physical and psychological health due to administration of excessive dosage of anesthetic gas is also mitigated. Therefore, the hollow fiber membrane is particularly suitable for human blood oxygenation comprising an anesthetic gas.

As a further improvement provided by the present disclosure, the hollow fiber membrane is used for gas-liquid separation.

In many occasions, it is not only required to perform liquid-liquid separation, but also required to perform gas-liquid separation. The hollow fiber membrane according to the present disclosure is also suitable for gas-liquid separation, particularly suitable for water-gas separation; this is because the outer surface of the hollow fiber membrane according to the present disclosure has a strong hydrophobic property such that water cannot permeate through, but gas can, thereby achieving the objective of gas-water separation.

The present disclosure achieves the following benefits: the asymmetric hydrophobic polyolefin hollow fiber membrane according to the present disclosure comprises a support layer and a separation layer, the separation layer comprising an outer surface, the outer surface comprising a quantity of first pores with a certain pore size. Presence of the first pores facilitates an anesthetic gas such as sevoflurane, xenon, remifentanil, or propofol to permeate through the hollow fiber membrane into the patient's blood, allowing for the patient to maintain sedated throughout a surgical process; meanwhile, presence of the first pores facilitates reduction of dosage of the anesthetic during the surgical process, which reduces the surgical cost and avoids secondary impairments to the patient due to application of excessive dosage of the anesthetic. In addition, the hollow fiber membrane further has a relatively long plasma permeation duration, a high tensile strength, and a high elongation at break, so as to satisfy application needs; therefore, the hollow fiber membrane is particularly suitable for human blood oxygenation comprising an anesthetic gas and for gas-liquid separation areas. In addition, the present disclosure further provides a method of preparing a hollow fiber membrane, which is quick, efficient, easily manipulated, and suitable for large-scale promotion.

### BRIEF DESCRIPTION OF THE DRAWINGS

FIG. 1 is a scanning electron microscope SEM image of the longitudinal section proximal to the outer surface side of a hollow fiber membrane prepared according to Example 1, wherein the magnification is 20000×;

FIG. 2 is a further magnified SEM image of the longitudinal section proximal to the outer surface side of the hollow fiber membrane prepared according to Example 1, wherein the magnification is 50000×;

FIG. 3 is a SEM image of the outer surface of the hollow fiber membrane prepared according to Example 1, wherein the magnification is 20000×;

FIG. 4 is a further magnified SEM image of the outer surface of the hollow fiber membrane prepared according to Example 1, wherein the magnification is 50000×;

FIG. 5 is a SEM image of an inner surface of the hollow fiber membrane prepared according to Example 2, wherein the magnification is 20000×;

FIG. 6 is a further magnified SEM image of the inner surface of the hollow fiber membrane prepared according to Example 1, wherein the magnification is 50000×;

FIG. 7 is a SEM image of a longitudinal section proximal to the outer surface of a hollow fiber membrane prepared according to Example 4, wherein the magnification is 20000×;

FIG. 8 is a further magnified SEM image of the longitudinal section proximal to the outer surface of the hollow fiber membrane prepared according to Example 4, wherein the magnification is 50000×;

FIG. 9 is a SEM image of the outer surface of the hollow fiber membrane prepared according to Example 4, wherein the magnification is 20000×;

FIG. 10 is a further magnified SEM image of the hollow fiber membrane prepared according to Example 4, wherein the magnification is 50000×;

FIG. 11 is a SEM image of an inner surface of the hollow fiber membrane prepared according to Example 4, wherein the magnification is 20000×;

FIG. 12 is a further magnified SEM image of the inner surface of the hollow fiber membrane prepared according to Example 4, wherein the magnification is 50000×.

### DETAILED DESCRIPTION

Hereinafter, the present disclosure will be further detailed through embodiments with reference to the accompanying drawings.

### Example 1

A method of preparing an asymmetric hydrophobic polyolefin hollow fiber membrane, comprising steps of:

Step 1: feeding 40 wt. % polypropylene into a twin-screw extruder, heating till being plasticized, followed by adding a solvent system comprising 45 wt. % methyl-12-hydroxystearate and 15 wt. % dioctyl adipate to form a mixture, and then stirring and mixing under 230° C. to obtain a homogeneous casting solution;

Step 2: extruding the casting solution out of a die with a temperature of 215° C. to obtain a molding having an inner surface and an outer surface;

Step 3: placing the molding in an air section for preliminary phase separation for 10 ms; wherein the air section has a temperature of 100° C. and a relative humidity of 30%;

Step 4: cooling the molding with a coolant comprising methyl-12-hydroxystearate at a cooling temperature of 40° C. for 55 ms;

Step 5: then, quenching the molding with a quenchant comprising methyl-12-hydroxystearate at a quenching temperature of 60° C. for 4 h, whereby a nascent membrane is obtained upon end of the quenching;

Step 6: extracting the nascent membrane for 24 h with 60° C. isopropanol to remove compound A and compound B, whereby a prototype membrane is obtained;

Step 7: subjecting the prototype membrane to high-temperature setting under 150° C., and stretching by 3% to relieve stress, thereby obtaining a finished membrane.

### Example 2

A method of preparing an asymmetric hydrophobic polyolefin hollow fiber membrane, comprising steps of:

Step 1: feeding 31 wt. % polypropylene into a twin-screw extruder, heating till being plasticized, followed by adding a solvent system comprising 46 wt. % dibutyl sebacate and 23 wt. % castor oil to form a mixture, and then stirring and mixing under 255° C. to obtain a homogeneous casting solution;

Step 2: extruding the casting solution out of a die with a temperature of 230° C. to obtain a molding having an inner surface and an outer surface;

Step 3: placing the molding in an air section for preliminary phase separation for Sins; wherein the air section has a temperature of 105° C. and a relative humidity of 25%;

Step 4: cooling the molding with a coolant comprising the solvent system (46 wt. % dibutyl sebacate and 23 wt. % castor oil) used when preparing the casting solution at a cooling temperature of 50° C. for 35 ms;

Step 5: then, quenching the molding with a quenchant comprising the solvent system (46 wt. % dibutyl sebacate and 23 wt. % castor oil) used when preparing the casting solution at a quenching temperature of 55° C. for 3 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 140° C., and stretching by 5% to relieve stress, thereby obtaining a finished membrane.

### Example 3

A method of preparing an asymmetric hydrophobic polyolefin hollow fiber membrane, comprising steps of:

Step 1: feeding 48 wt. % polypropylene into a twin-screw extruder, heating till being plasticized, followed by adding a solvent system comprising 39 wt. % dibutyl phthalate and 13 wt. % dioctyl adipate to form a mixture, and then stirring and mixing under 245° C. to obtain a homogeneous casting solution;

Step 2: extruding the casting solution out of a die with a temperature of 220° C. to obtain a molding having an inner surface and an outer surface;

Step 3: placing the molding in an air section for preliminary phase separation for 15 ms; wherein the air section has a temperature of 80° C. and a relative humidity of 35%;

Step 4: cooling the molding with a coolant comprising dibutyl phthalate at a cooling temperature of 30° C. for 60 ms;

Step 5: then, quenching the molding with a quenchant comprising dibutyl phthalate at a quenching temperature of 50° C. for 5 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 170° C., and stretching by 2% to relieve stress, thereby obtaining a finished membrane.

### Example 4

A method of preparing an asymmetric hydrophobic polyolefin hollow fiber membrane, comprising steps of:

Step 1: feeding 45 wt. % polypropylene into a twin-screw extruder, heating till being plasticized, followed by adding a solvent system comprising 40 wt. % methyl-12-hydroxystearate and 15 wt. % dimethyl phthalate to form a mixture, and then stirring and mixing under 235° C. to obtain a homogeneous casting solution;

Step 2: extruding the casting solution out of a die with a temperature of 220° C. to obtain a molding having an inner surface and an outer surface;

Step 3: placing the molding in an air section for preliminary phase separation for 8 ms; wherein the air section has a temperature of 110° C. and a relative humidity of 20%; then, precooling the molding with a coolant comprising the solvent system (comprising 40 wt. % methyl-12-hydroxystearate and 15 wt. % dimethyl phthalate) used when preparing the casting solution at a precooling temperature of 140° C. for 6 ms;

Step 4: cooling the molding with the coolant comprising the solvent system (comprising 40 wt. % methyl-12-hydroxystearate and 15 wt. % dimethyl phthalate) used when preparing the casting solution at a cooling temperature of 40° C. for 60 ms;

Step 5: then, quenching the molding with a quenchant comprising the solvent system (comprising 40 wt. % methyl-12-hydroxystearate and 15 wt. % dimethyl phthalate) used when preparing the casting solution at a quenching temperature of 70° C. for 4 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 160° C., and stretching by 1% to relieve stress, thereby obtaining a finished membrane.

### Example 5

A method of preparing an asymmetric hydrophobic polyolefin hollow fiber membrane, comprising steps of:

Step 1: feeding 20 wt. % polypropylene and 20 wt. % polyethylene into a twin-screw extruder, heating till being plasticized, followed by adding a solvent system comprising 40 wt. % dibutyl sebacate and 20 wt. % palm oil, and then stirring and mixing under 245° C. to obtain a homogeneous casting solution;

Step 2: extruding the casting solution out of a die with a temperature of 220° C. to obtain a molding having an inner surface and an outer surface;

Step 3: placing the molding in an air section for preliminary phase separation for 6 ms; wherein the air section has a temperature of 80° C. and a relative humidity of 35%;

Step 4: cooling the molding with a coolant comprising dibutyl sebacate at a cooling temperature of 35° C. for 50 ms;

Step 5: then, quenching the molding with a quenchant comprising dibutyl sebacate at a quenching temperature of 55° C. for 4.5 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 145° C., and stretching by 6% to relieve stress, thereby obtaining a finished membrane.

### Example 6

A method of preparing an asymmetric hydrophobic polyolefin hollow fiber membrane, comprising steps of:

Step 1: feeding 30 wt. % poly (4-methyl-1-pentene) (PMP) and 10 wt. % polyethylene into a twin-screw extruder, heating till being plasticized, followed by adding a solvent system comprising 45 wt. % dehydrated castor oil fatty acid and 15 wt. % dioctyl adipate to form a mixture, and then stirring and mixing under 240° C. to obtain a homogeneous casting solution;

Step 2: extruding the casting solution out of a die with a temperature of 215° C. to obtain a molding having an inner surface and an outer surface;

Step 3: placing the molding in an air section for preliminary phase separation for 7 ms; wherein the air section has a temperature of 60° C. and a relative humidity of 10%;

Step 4: cooling the molding with a coolant comprising the solvent system (comprising 45 wt. % dehydrated castor oil fatty acid and 15 wt. % dioctyl adipate) used when preparing the casting solution at a cooling temperature of 25° C. for 75 ms;

Step 5: then, quenching the molding with a quenchant comprising the solvent system (comprising 45 wt. % dehydrated castor oil fatty acid and 15 wt. % dioctyl adipate) used when preparing the casting solution at a quenching temperature of 45° C. for 5 h, whereby a nascent membrane is obtained upon end of the quenching;

Step 6: extracting the nascent membrane for 24 h with 65° C. isopropanol to remove compound A and compound B, whereby a prototype membrane is obtained;

### Example 7

A method of preparing an asymmetric hydrophobic polyolefin hollow fiber membrane, comprising steps of:

Step 1: feeding 30 wt. % poly (4-methyl-1-pentene) (PMP) and 10 wt. % polypropylene into a twin-screw extruder, heating till being plasticized, followed by adding a solvent system comprising 40 wt. % dehydrated castor oil fatty acid and 20 wt. % mineral oil to form a mixture, and then stirring and mixing under 250° C. to obtain a homogeneous casting solution;

Step 2: extruding the casting solution out of a die with a temperature of 225° C. to obtain a molding having an inner surface and an outer surface;

Step 3: placing the molding in an air section for preliminary phase separation for 14 ms; wherein the air section has a temperature of 130° C. and a relative humidity of 20%;

Step 4: cooling the molding with a coolant comprising dehydrated castor oil fatty acid at a cooling temperature of 35° C. for 50 ms;

Step 5: then, quenching the molding with dehydrated castor oil fatty acid as quenchant at a quenching temperature of 45° C. for 3.5 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 170° C. for high-temperature setting, and stretching by 6% to relieve stress, thereby obtaining a finished membrane.

### Example 8

A method of preparing an asymmetric hydrophobic polyolefin hollow fiber membrane, comprising steps of:

Step 1: feeding 20 wt. % poly (4-methyl-1-pentene) (PMP) and 20 wt. % polypropylene into a twin-screw extruder, heating till being plasticized, followed by adding a solvent system comprising 40 wt. % methyl-12-hydroxystearate and 20 wt. % dimethyl phthalate to form a mixture, and then stirring and mixing under 240° C. to obtain a homogeneous casting solution;

Step 2: extruding the casting solution out of a die with a temperature of 220° C. to obtain a molding having an inner surface and an outer surface;

Step 3: placing the molding in an air section for preliminary phase separation for 12 ms; wherein the air section has a temperature of 120° C. and a relative humidity of 30%; then, precooling the molding with a coolant comprising the solvent system (comprising 40 wt. % methyl-12-hydroxystearate and 20 wt. % dimethyl phthalate) used when preparing the casting solution at a precooling temperature of 130° C. for 5 ms;

Step 4: cooling the molding with the coolant comprising the solvent system (comprising 40 wt. % methyl-12-hydroxystearate and 20 wt. % dimethyl phthalate) used when preparing the casting solution at a cooling temperature of 45° C. for 65 ms;

Step 5: then, quenching the molding with a quenchant comprising the solvent system (comprising 40 wt. % methyl-12-hydroxystearate and 20 wt. % dimethyl phthalate) used when preparing the casting solution at a quenching temperature of 65° C. for 4 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 150° C. for high-temperature setting, and stretching by 1% to relieve stress, thereby obtaining a finished membrane.

### Comparative Example 1

A method of preparing an asymmetric hydrophobic polyolefin hollow fiber membrane, comprising steps of:

Step 1: feeding 40 wt. % polypropylene into a twin-screw extruder, heating till being plasticized, followed by adding a solvent system comprising 45 wt. % methyl-12-hydroxystearate and 15 wt. % dioctyl adipate to form a mixture, and then stirring and mixing under 230° C. to obtain a homogeneous casting solution;

Step 2: extruding the casting solution out of a die with a temperature of 215° C. to obtain a molding having an inner surface and an outer surface;

Step 3: placing the molding in an air section for preliminary phase separation for 10 ms; wherein the air section has a temperature of 100° C. and a relative humidity of 30%;

Step 4: cooling the molding with a coolant comprising dioctyl adipate at a cooling temperature of 40° C. for 55 ms;

Step 5: then, quenching the molding with a quenchant comprising dioctyl adipate at a quenching temperature of 60° C. for 4 h, whereby a nascent membrane is obtained upon end of the quenching;

Step 6: extracting the nascent membrane for 24 h with 65° C. isopropanol to remove compound A and compound B, whereby a prototype membrane is obtained;

Step 7: subjecting the prototype membrane to high-temperature setting under 150° C., and stretching by 3% to relieve stress, thereby obtaining a finished membrane.

### Structural and Performance Testing for Test Samples

1. Structural Characterization. The membrane body structure of each test sample is subjected to morphology characterization using a SEM (Hitachi S-5500) to obtain relevant data. Specific results are set forth in the table below.

| | | Overall | | Separation- |
|---|---|---|---|---|
| Test | Inner | Membrane | Volumetric | layer |
| Samples | Diameter/μm | Thickness/μm | porosity/% | thickness/μm |
| Example 1 | 195.6 | 40.3 | 46 | 1.1 |
| Example 2 | 150.2 | 32.5 | 54 | 0.6 |
| Example 3 | 261.7 | 48.2 | 41 | 1.9 |
| Example 4 | 232.4 | 44.6 | 48 | 1.6 |
| Example 5 | 217.3 | 38.4 | 51 | 0.8 |
| Example 6 | 172.9 | 41.5 | 44 | 1.4 |
| Example 7 | 206.1 | 39.2 | 37 | 0.9 |
| Example 8 | 192.8 | 40.8 | 40 | 1.2 |
| Comparative | 209.4 | 43.1 | 32 | 2.2 |
| Example 1 | | | | |
| | Proportion of | | | |
| | separation- | | | |
| | layer thickness | | | |
| | to the total | Mean | | Mean |
| | thickness | pore size | | pore |
| | of the | of the | Thickness | size of |
| Test | hollow fiber | separation | of transition | transition |
| Samples | membrane/% | layer/nm | layer/nm | layer/nm |
| Example 1 | 2.73% | 36 | \ | \ |
| Example 2 | 1.85% | 23 | \ | \ |
| Example 3 | 3.94% | 51 | \ | \ |
| Example 4 | 3.59% | 43 | 42 | 237 |
| Example 5 | 2.08% | 28 | \ | \ |
| Example 6 | 3.37% | 39 | \ | \ |
| Example 7 | 2.30% | 31 | \ | \ |
| Example 8 | 2.94% | 34 | 27 | 164 |
| Comparative | 5.10% | \ | \ | \ |
| example 1 | | | | |

Examples 1-3, Examples 5-7 and Comparative Example 1 do not comprise a precooling step in preparing a hollow fiber membrane, such that the resultant hollow fiber membranes do not have a transition layer; Examples 4 and 8 comprise a precooling step in preparing a hollow fiber membrane, such that the resultant hollow fiber membranes have a transition layer; in addition, compared with Example 1, Comparative Example 1 uses a non-solvent compound B as the coolant, which leads to a too fast curing speed for the separated phase, resulting in a dense separation layer for the resultant hollow fiber membrane. Without pores at the outer surface of the hollow fiber membrane, an anesthetic gas cannot permeate through the hollow fiber membrane prepared according to Comparative Example 1.

| | Pore size range | Pore size range | |
|---|---|---|---|
| | of the first | of the first | Pore density |
| | pores at the | pores at the | of the first |
| | outer surface | outer surface | pores at the |
| Test | in the first | in the second | outer surface |
| Samples | direction/nm | direction/nm | (pores/1 μm²) |
| Example 1 | 180-270 | 15-70 | 22 |
| Example 2 | 100-250 | 12-50 | 31 |
| Example 3 | 200-290 | 20-85 | 13 |
| Example 4 | 190 = 280 | 18-75 | 16 |
| Example 5 | 160-250 | 10-65 | 29 |
| Example 6 | 195-285 | 25-100 | 15 |
| Example 7 | 185-280 | 14-60 | 26 |
| Example 8 | 180-295 | 17-75 | 20 |
| Comparative | \ | \ | \ |
| Example 1 | | | |

The table unveils that the hollow fiber membranes prepared in Examples 1-8 of the disclosure all have a quantity of first pores with a certain pore size at their respective outer surface, which facilitates permeation of the anesthetic gas without affecting plasma permeation duration, such that the resultant hollow fiber membranes are particularly suitable for blood oxygenation comprising an anesthetic gas; in contrast, the hollow fiber membrane prepared according to Comparative Example 1 does not have pores at its outer surface, such that the anesthetic gases cannot permeate through the resultant hollow fiber membrane.

### 2. Property Testing

Tensile Strength and Elongation at Break Testing: each sample is stretched using a stretcher at a uniform speed under room temperature (where the stretching velocity is 50 mm/min and the distance between the upper and lower fixtures is 30 mm) till being broken, whereby the tensile strength and the elongation at break are measured. The testing is repeated three times. The mean value of the measured tensile strengths and the mean value of the measured elongations at break are computed as the final tensile strength value and the final elongation at break of the membrane.

| | Surface Energy | | |
|---|---|---|---|
| | of the Outer | | |
| | Surface of Test | | |
| Test | Sample under | Tensile | Elongation |
| Samples | 20° C. (mN/m) | Strength/CN | at Break/% |
| Example 1 | 32 | 181 | 214 |
| Example 2 | 34 | 188 | 235 |
| Example 3 | 33 | 172 | 243 |
| Example 4 | 31 | 140 | 276 |
| Example 5 | 36 | 200 | 187 |
| Example 6 | 26 | 110 | 452 |
| Example 7 | 28 | 230 | 161 |
| Example 8 | 24 | 160 | 268 |
| Comparative | 34 | 190 | 201 |
| Example 1 | | | |

Surface Energy Testing: the surface energy testing for the outer surface of each hollow fiber membrane is performed as such using a Dyne pen under 20° C.: drawing the Dyne pen over the outer surface of the hollow fiber membrane in a 10 cm-long ink pass, and observing whether over 90% of the ink pass has been drawn back into droplets in less than 2 s till the ink pass stops drawing back and droplets appear; the measured surface energy of the ink is the surface energy of the outer surface of the membrane.

The table unveils that the hollow fiber membranes prepared according to Examples 1-8 all have a relatively high tensile strength and a relatively high elongation at break, which may satisfy industrial requirements; meanwhile, the hollow fiber membranes have a strong hydrophobic property.

The hollow fiber membranes resulting from Examples 1-8 are subjected to gas permeation rate testing. The testing manner is described as follows:

One side of each membrane sample of 0.1 m² is subjected to a to-be-tested gas (oxygen, carbon dioxide, and anesthetic gases) at the temperature of 25° C. under 1bar; the to-be-tested gas is charged into the lumen of the hollow fiber membrane; the volumetric flow rate of the gas permeated through the membrane wall of the test sample is measured using a flowmeter (Japan KOFLOC/4800); the testing is repeated three times from the inside of the membrane to the outside of the membrane and three times from the outside of the membrane to the inside of the membrane; the mean value of the six measurements is computed as the gas permeation rate of the membrane.

Unit of the Gas Permeation Rate: L/(min·bar·m²)

| | | | | Gas | Gas |
|---|---|---|---|---|---|
| | | | Gas | Separation | Separation |
| | O₂ | CO₂ | Separation | Factor α | Factor α |
| Test | Permeation | Permeation | Factor α | (O₂/ | (O₂/ |
| Sample | Rate | Rate | (CO₂/O₂) | sevoflurane) | remifentanil) |
| Example | 25 | 42 | 1.68 | 320 | 450 |
| 1 | | | | | |
| Example | 30 | 52 | 1.73 | 277 | 442 |
| 2 | | | | | |
| Example | 28 | 47 | 1.68 | 362 | 492 |
| 3 | | | | | |
| Example | 20 | 36 | 1.80 | 285 | 412 |
| 4 | | | | | |
| Example | 16 | 31 | 1.94 | 306 | 395 |
| 5 | | | | | |
| Example | 7 | 15 | 2.14 | 261 | 368 |
| 6 | | | | | |
| Example | 10 | 22 | 2.20 | 293 | 421 |
| 7 | | | | | |
| Example | 12 | 27 | 2.25 | 304 | 437 |
| 8 | | | | | |

The table unveils that the hollow fiber membranes prepared according to Examples 1-8 all have a relatively high oxygen permeation rate and a relatively high carbon dioxide permeation rate, which facilitates quick discharge of the carbon dioxide out of the blood and facilitates quick permeation of the oxygen through the hollow fiber membranes into the blood; meanwhile, the anesthetic gases may permeate through the hollow fiber membranes into the patient's blood at a certain permeation rate, such that the patent maintains sedated throughout a surgical process, thereby ensuring smooth proceeding of the surgery.

### Testing on Plasma Permeation Duration of the Hollow Fiber Membranes

The plasma permeation duration of each test sample is measured by: letting a 37° C. phospholipid solution (1.5 g/L-α-lecithin dissolved in 500 ml normal saline solution) flow across the surface of each membrane sample at 61/(min*m²) under 1.0bar; letting air flow across the opposite side of the membrane sample and then pass through a cold trap; measuring the weight of the liquid aggregated in the cold trap as a function of time. The duration till noticeable increase of the weight, i.e., till the liquid is first noticeably aggregated in the cold trap, is determined as the plasma permeation duration.

The testing unveils that the plasma permeation durations of the hollow fiber membranes prepared according to Examples 1-8 are all over 48 hours, indicating that the hollow fiber membranes prepared according to the present disclosure have a very long service life and may ensure smooth proceeding of a surgery.

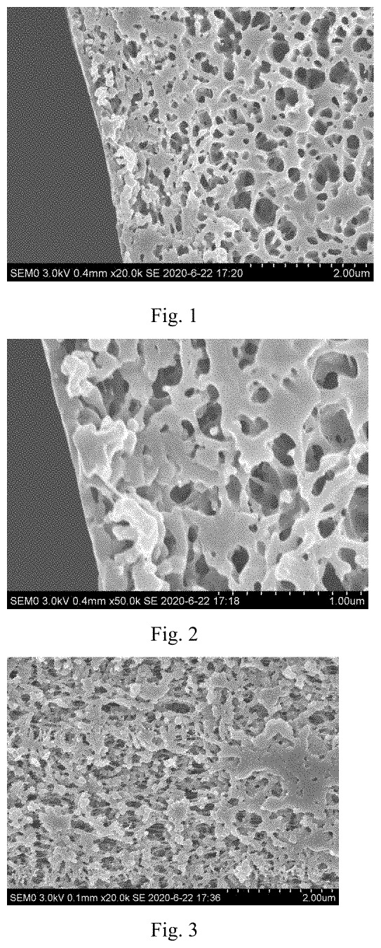

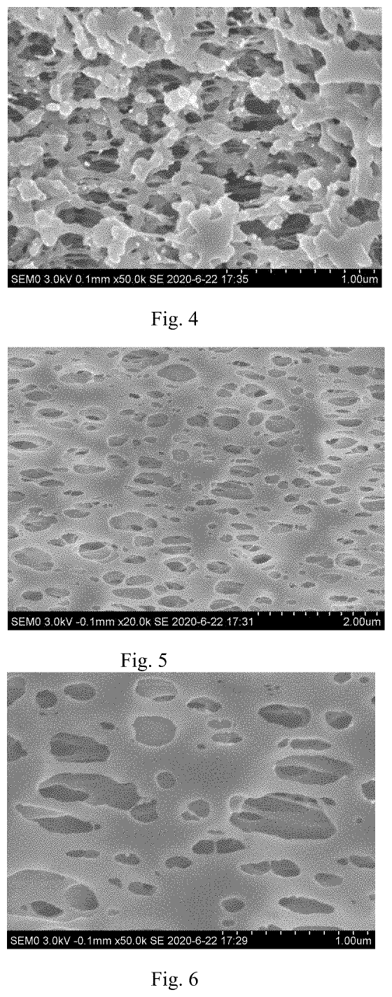

FIGS. 1-6 are SEM images of the hollow fiber membrane prepared according to Example 1, which unveil that the separation layer of the hollow fiber membrane prepared according to Example 1 is porous, and the outer surface of the hollow fiber membrane has a quantity of first pores with a certain pore size, which facilitate permeation of an anesthetic gas.

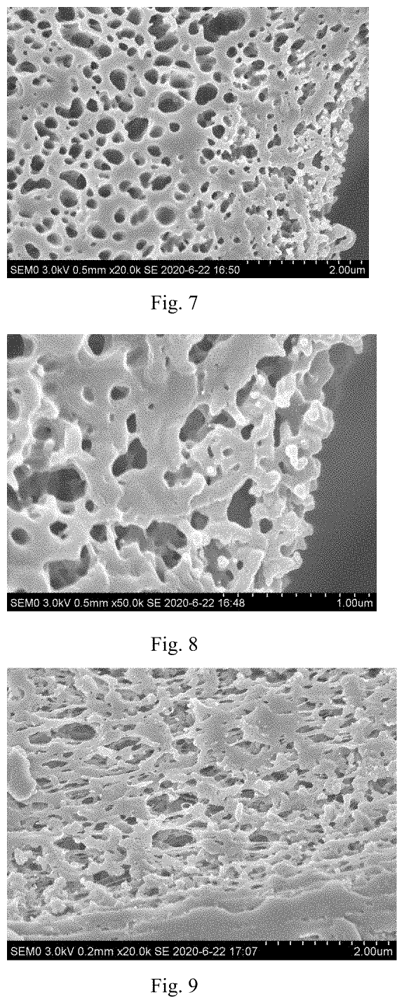

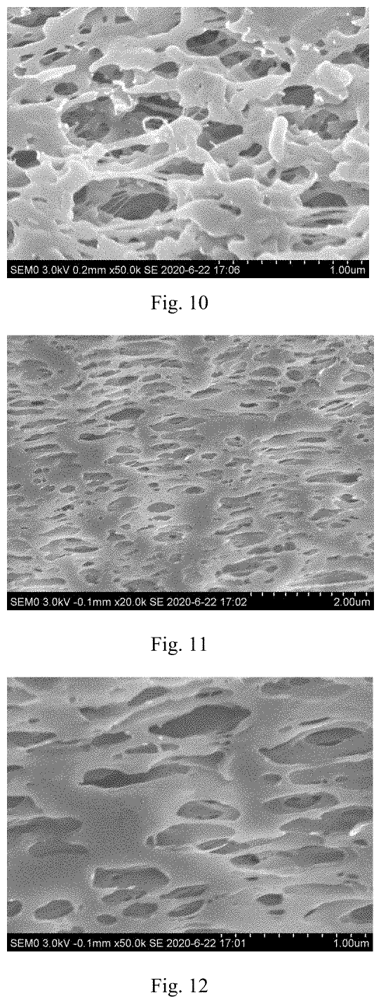

FIGS. 7-12 are SEM images of the hollow fiber membrane prepared according to Example 4, which unveil that the separation layer of the hollow fiber membrane prepared according to Example 4 is porous, and the outer surface of the hollow fiber membrane has a quantity of first pores with a certain pore size, which facilitate permeation of an anesthetic gas.

The hollow fiber membranes prepared according to the disclosure are particularly suitable for blood oxygenation comprising an anesthetic and also suitable for gas-liquid separation.

What have been described above are only preferred embodiments of the disclosure. The protection scope of the disclosure is not limited to the examples described above, and any technical solution under the idea of the disclosure falls within the protection of the disclosure. It is noted that to those improvements and modifications made by a person of normal skill in the art without departing from the principle of the disclosure shall also be deemed as falling within the protection scope of the disclosure.

1. An asymmetric hydrophobic polyolefin hollow fiber membrane, comprising a support layer and a separation layer, the support layer comprising an inner surface facing a lumen of the hollow fiber membrane, the separation layer comprising an outer surface, the outer surface being located at the side of the separation layer opposite the support layer, wherein:
the outer surface comprises a plurality of first pores, the first pores having a pore size of 10 nm to 300 nm in a first direction of the outer surface and a pore size of 10 nm to 300 nm in a second direction of the outer surface; wherein the first direction of the outer surface is parallel to an axial direction of the hollow fiber membrane, and the second direction of the outer surface is parallel to a radial direction of the hollow fiber membrane
the first pores at the outer surface have a pore density of 4 to 45 pores/1 μm²; the outer surface of the hollow fiber membrane has a surface energy of 10 mN/m to 45 mN/m under 20° C.; and
the hollow fiber membrane has a tensile strength of at least 100CN and an elongation at break of at least 150%.

2. The asymmetric hydrophobic polyolefin hollow fiber membrane according to claim 1, wherein the first pores have a pore size of 150 nm to 300 nm in the first direction of the outer surface, the first pores have a pore size of 10 nm to 90 nm in the second direction of the outer surface; wherein the first direction is parallel to the axial direction of the hollow fiber membrane, and the second direction is parallel to the radial direction of the hollow fiber membrane; and
the first pores have a pore density of 4 to 35 pores/1 μm².

3. The asymmetric hydrophobic polyolefin hollow fiber membrane according to claim 1, wherein the separation layer has a thickness of 0.1 μm to 2 μm, accounting for 0.5 to 5% of total thickness of the hollow fiber membrane.

4. The asymmetric hydrophobic polyolefin hollow fiber membrane according to claim 1, wherein the separation layer is porous, and a mean pore size of the separation layer is 10 nm to 60 nm.

5. The asymmetric hydrophobic polyolefin hollow fiber membrane according to claim 1, wherein the hollow fiber membrane has an O₂ permeation rate of 1 to 50 L/min·bar·m²; the hollow fiber membrane has a gas separation factor α of 1 to 4 between CO₂ and O₂ and a gas separation factor α of at least 150 between O₂ and an anesthetic gas.

6. The asymmetric hydrophobic polyolefin hollow fiber membrane according to claim 5, wherein the hollow fiber membrane has an O₂ permeation rate of 10 to 40 L/min·bar·m² and a CO₂ permeation rate of 15 to 80 L/min·bar·m².

7. The asymmetric hydrophobic polyolefin hollow fiber membrane according to claim 5, wherein the hollow fiber membrane has a gas separation factor α of at least 200 between O₂ and the anesthetic gas, wherein the anesthetic gas is selected from the group consisting of at least one of sevoflurane, xenon, remifentanil, and propofol.

8. The asymmetric hydrophobic polyolefin hollow fiber membrane according to claim 1, wherein the hollow fiber membrane has a plasma permeation duration of at least 48 h.

9. The asymmetric hydrophobic polyolefin hollow fiber membrane according to claim 1, further comprising a transition layer disposed between the support layer and the separation layer, the transition layer having a thickness of 10 nm to 50 nm and a mean pore size of 100 nm to 300 nm.

10. The asymmetric hydrophobic polyolefin hollow fiber membrane according to claim 1, wherein the hollow fiber membrane has a thickness of 30 μm to 50 μm and an inner diameter of 100 μm to 300 μm; and the hollow fiber membrane has a volumetric porosity of 30% to 60%.

11. A method of preparing the asymmetric hydrophobic polyolefin hollow fiber membrane according to claim 1, comprising:
step 1: heating to plasticize a polyolefin polymer comprising only carbon and hydrogen, followed by dissolving the plasticized polyolefin polymer in a solvent system comprising compound A and compound B, and mixing under an environment higher than a critical delamination temperature, resulting in a homogeneous casting solution, wherein compound A is a solvent for polyolefin polymer, and compound B is a non-solvent for polyolefin polymer and elevates separation temperature for a phase comprising the polyolefin polymer and the compound A; the solvent system has a range exhibiting a homogeneous solution at an elevated temperature, a critical delamination temperature upon cooling, a miscibility gap in a liquid state of aggregation lower than the critical delamination temperature, and a cold curing temperature;
step 2: extruding the casting solution in a die having a temperature higher than the critical delamination temperature to form a molding having an inner surface and an outer surface;
step 3: placing the molding in an air section for preliminary phase separation;
step 4: cooling the molding with a coolant comprising compound A at a cooling temperature of 5° C. to 60° C. for 20 ms to 75 ms;
step 5: quenching the molding with a quenchant comprising compound A at a quenching temperature of 40° C. to 80° C. for 2 h to 5 h, whereby a nascent membrane is obtained upon end of the quenching;
step 6: removing compound A and compound B from the nascent membrane to obtain a prototype membrane.

12. The method of preparing the asymmetric hydrophobic polyolefin hollow fiber membrane according to claim 11, wherein the polyolefin polymer is selected from the group consisting of at least one of polyethylene, polypropylene, and poly(4-methyl-1-pentene); and a concentration of the polyolefin polymer in the casting solution is 30% to 50%.

13. The method of preparing the asymmetric hydrophobic polyolefin hollow fiber membrane according to claim 11, wherein the compound A is selected from the group consisting of one or more of dehydrated castor oil fatty acid, methyl-12-hydroxystearate, paraffin oil, dibutyl sebacate, and dibutyl phthalate; the compound B is selected from the group consisting of one or more of dioctyl adipate, castor oil, mineral oil, palm oil, rapeseed oil, olive oil, dimethyl phthalate, dimethyl carbonate, and glyceryl triacetate; and a mass ratio of compound A to compound B is 1-5:1.

14. The method of preparing the asymmetric hydrophobic polyolefin hollow fiber membrane according to claim 11, wherein in step 3, the molding stays in the air section for 1.5 ms to 20 ms; the air section has a temperature of 50° C. to 150° C. and a relative humidity of not greater than 50%.

15. The method of preparing the asymmetric hydrophobic polyolefin hollow fiber membrane according to claim 11, wherein before the molding is subjected to cooling treatment in step 4, the molding resultant from the preliminary phase separation in step 3 is pre-cooled with a treating solution comprising compound A at a precooling temperature ranging from 120° C. to 160° C. for a precooling duration of 2 ms to 10 ms.

16. The method of preparing the asymmetric hydrophobic polyolefin hollow fiber membrane according to claim 11, wherein after the prototype membrane is obtained in step 5, the prototype membrane is placed under an environment of 120° C. to 180° C. for high-temperature setting and stretched by 0.5% to 10% to relieve stress, whereby a finished membrane is obtained.

17. A method comprising using the asymmetric hydrophobic polyolefin hollow fiber membrane according to claim 1 for human blood oxygenation comprising an anesthetic gas.

18. A method comprising using the asymmetric hydrophobic polyolefin hollow fiber membrane according to claim 1 for gas-liquid separation.
